(19) United States
(12) Patent Application Publication
(10) Pub. No.: US 2015/0072378 A1
(43) Pub. Date: Mar. 12, 2015

(54) Systems and Methods for Incubating Samples

(71) Applicant: Abbott Laboratories, Abbott Park, IL (US)

(72) Inventor: Hidesuke Kokawa, Sunnyvale, CA (US)

(21) Appl. No.: 14/480,466

(22) Filed: Sep. 8, 2014

Related U.S. Application Data
(60) Provisional application No. 61/875,417, filed on Sep. 9, 2013.

(51) Int. Cl. G01N 1/16; G01N 33/49; G01N 1/38

(52) U.S. Cl. CPC G01N 1/16; G01N 1/38; G01N 33/492; G01N 2001/1031
USPC 435/39; 435/286.2

(57) ABSTRACT

Provided are incubation systems for incubating samples for an assay. In certain aspects, the systems include a rotary platform, a sample container positioned at a peripheral edge of the platform, a plurality of activity sites including at least a first and a second activity site, a processor, and a memory that includes instructions. When executed by the processor, the instructions cause the system to rotate the rotary platform to move the sample container from the first activity site to the second activity site, extract a first portion of the sample from the sample container at the first activity site for a measurement based on a first incubation time period, and extract a second portion of the sample from the sample container at the second activity site for a measurement based on a second incubation time period, the second incubation time period being longer than the first incubation time period. Methods for incubating samples, e.g., methods which employ the systems of the present disclosure, are also provided.

### CROSS-REFERENCE

This application claims the benefit of U.S. Provisional Patent Application No. 61/875,417 filed Sep. 9, 2013, which application is incorporated herein by reference in its entirety.

### SUMMARY

Provided are incubation systems for incubating samples for an assay. In certain aspects, the systems include a rotary platform, a sample container positioned at a peripheral edge of the platform, a plurality of activity sites including at least a first and a second activity site, a processor, and a memory that includes instructions. When executed by the processor, the instructions cause the system to rotate the rotary platform to move the sample container from the first activity site to the second activity site, extract a first portion of the sample from the sample container at the first activity site for a measurement based on a first incubation time period, and extract a second portion of the sample from the sample container at the second activity site for a measurement based on a second incubation time period, the second incubation time period being longer than the first incubation time period. Methods for incubating samples, e.g., methods which employ the systems of the present disclosure, are also provided.

### BRIEF DESCRIPTION OF THE FIGURES

The accompanying drawings, which are incorporated herein, form part of the specification. Together with this written description, the drawings further serve to explain the principles of, and to enable a person skilled in the relevant art(s), to make and use the systems and methods of the present disclosure. In the drawings, like reference numbers indicate identical or functionally similar elements.

FIG. 1 illustrates a one-dilution incubating system for incubating samples for a hematology assay, according to one embodiment;

FIG. 2 illustrates a time diagram for an example method in two assays may be performed by a single optical bench;

FIG. 3 illustrates a diagram of an example system including the incubation system described in FIG. 1, according to one embodiment;

FIG. 4 illustrates a block diagram for an exemplary system including the incubation system described in FIG. 1, according to one embodiment;

FIG. 5 illustrates a system with the rotary platform described in FIG. 1 as well as additional sampling containers for additional measurement;

FIG. 6 illustrates a one-dilution incubation system, according to certain embodiments;

FIG. 7 illustrates a three-dilution incubation system, according to certain embodiments;

FIG. 8 illustrates the incubation system of FIG. 7, according to one embodiment; and

FIG. 9 illustrates a block diagram of an example data processing device 405 of FIG. 1, according to one embodiment.

### DETAILED DESCRIPTION

Provided are incubation systems for incubating samples for an assay. In certain aspects, the systems include a rotary platform, a sample container positioned at a peripheral edge of the platform, a plurality of activity sites including at least a first and a second activity site, a processor, and a memory that includes instructions. When executed by the processor, the instructions cause the system to rotate the rotary platform to move the sample container from the first activity site to the second activity site, extract a first portion of the sample from the sample container at the first activity site for a measurement based on a first incubation time period, and extract a second portion of the sample from the sample container at the second activity site for a measurement based on a second incubation time period, the second incubation time period being longer than the first incubation time period. Methods for incubating samples, e.g., methods which employ the systems of the present disclosure, are also provided.

Before the systems and methods of the present disclosure are described in greater detail, it is to be understood that the systems and methods are not limited to particular embodiments described, as such may, of course, vary. It is also to be understood that the terminology used herein is for the purpose of describing particular embodiments only, and is not intended to be limiting, since the scope of the systems and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the systems and methods. The upper and lower limits of these smaller ranges may independently be included in the smaller ranges and are also encompassed within the systems and methods, subject to any specifically excluded limit in the stated range. Where the stated range includes one or both of the limits, ranges excluding either or both of those included limits are also included in the systems and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the systems and methods belong. Although any systems and methods and materials similar or equivalent to those described herein can also be used in the practice or testing of the systems and methods, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the systems and methods, which are, for clarity, described in the context of separate embodiments, may also be provided in combination in a single embodiment. Conversely, various features of the systems and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present systems and methods and are disclosed herein just as if each and every such sub-combination of chemical groups was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present systems and methods. Any recited method can be carried out in the order of events recited or in any other order which is logically possible.

### Systems

Aspects of the present disclosure include systems for incubating samples for an assay.

According to certain embodiments, the systems include a rotary platform, a sample container including a sample positioned at a peripheral edge of the platform, a plurality of activity sites including at least a first and a second activity site, a processor, and a memory including instructions. The instructions, when executed by the processor, cause the system to rotate the rotary platform to move the sample container from the first activity site to the second activity site, extract a first portion of the sample from the sample container at the first activity site for a measurement based on a first incubation time period, and extract a second portion of the sample from the sample container at the second activity site for a measurement based on a second incubation time period. The second incubation time period is longer than the first incubation time period.

Systems of the present disclosure include one or more activity sites (e.g., a plurality of activity sites) through which the sample container (e.g., a tube, vial (e.g., dilution vial), cuvette, mixing cup, or any other sample container of interest) is moved. Activity sites of interest include, but are not limited to, an activity site for providing a sample to a sample container, an activity site for diluting a sample (e.g., a hematology sample), an activity site for mixing a diluted sample, an activity site for incubating a sample, an activity site for washing a sample container, an activity site for drying a sample container, any other activity site useful for carrying out an assay, or any combination of such activity sites.

Various activities may be performed at an activity site depending on the application/assay of interest. In certain aspects, systems of the present disclosure include an activity site for disposing on the rotary platform a sample container which already includes a sample of interest. Alternatively, or additionally, the system may include an activity site for delivering a sample to an empty sample container (e.g., a new sample container or a previously used sample container that has been washed and/or dried) already present on the rotary platform. According to certain embodiments, the incubation system includes a sample delivery device operably positioned at the corresponding activity site to provide the sample within the sample container. Example sample delivery devices include, but are not limited to, a probe, pipettor, or any other device suitable for delivering a sample to the sample container. The delivery device may obtain a volume of sample from a sample source (e.g., a primary sample vial, test tube, or other container).

Systems of the present disclosure may include an activity site for diluting a sample (e.g., a hematology sample). The activity site for diluting a sample may be the same as, or different from, the activity site for providing a sample to a sample container. That is, a sample may be provided to a sample container and diluted at the same activity site or different activity sites. An activity site for diluting a sample may include a dilution device (e.g., a probe, pipettor, or the like) for diluting the sample at that activity site. The sample may be diluted using a diluent suitable for the particular assay of interest. Diluents may include a buffer (e.g., a phosphate or other suitable buffer), a salt, a surfactant, a dye (e.g., a fluorescent dye, such as a cell membrane-permeable and nucleic acid-binding fluorescent dye), an anti-microbial agent, a lysing reagent, any other diluent component useful for the assay of interest, or any combination thereof.

According to certain embodiments, systems of the present disclosure include an activity site for mixing a sample (e.g., a sample to which a diluent has been added) in the sample container. In certain aspects, the system may include a mixing device for mixing a diluted sample at the activity site for mixing a sample. Example mixing devices include, but are not limited to, a rotary stirrer, a vibration type mixer (e.g., a piezo vibrator, etc.), or a motor-driven mixer such as a paddle mixer, e.g., either a rotary paddle mixer, an “up-down” motion paddle mixer, or a combination thereof, for instance.

Systems of the present disclosure may include one or more (e.g., 1, 2, 3, or more) activity sites for incubating a sample in the sample container. The one or more activity sites for incubating a sample may overlap with one or more other activity sites in the system. For example, an activity site for incubating a sample may also be an activity site for diluting the sample, mixing the sample, extracting a portion of the sample, and/or the like. In certain aspects, the one or more activity sites for incubating the sample does not include a heating device. In other aspects, the one or more activity sites for incubating the sample include a heating device, e.g., for heating the sample to an incubation and/or assay temperature of interest. Any suitable heating device may be employed, including but not limited to, a hot air bath, a water bath, a heat block, a heating element positioned in sufficient proximity to the sample container to heat the sample, or the like. In certain aspects, the heating device includes a hot air bath, e.g., positioned beneath the rotary platform, above the rotary platform, or both.

Systems of the present disclosure may include one or more (e.g., 1, 2, 3, or more) activity stations for extracting all or a portion of a sample from the sample container for use in one or more assays of interest (e.g., one or more hematology assays of interest). Such an activity station(s) may include a sample extraction device, such as a pipetting device or suction probe for removing all or a portion of the sample and transferring the all or a portion of the sample to an assay container (e.g., a cuvette, test tube, or the like) or directly into an assay system, e.g., a flow cytometer or other system capable of performing an assay of interest on the sample. In certain aspects, the system includes an extraction device to extract a first portion of a sample from the sample container at the first activity site for a measurement based on a first incubation time period. Such a system may further include an extraction device to extract a second portion of the sample from the sample container at the second activity site for a measurement based on a second incubation time period. The same extraction device may be used to extract the first and second portions of the sample from the sample container, or two different extraction devices may be used.

As noted above, systems of the present disclosure may include an activity site for washing the sample container. An activity site for washing a sample container may be included in a subject system, e.g., to wash a sample container when a sample therein is no longer needed. A sample may no longer be needed when, e.g., one or more portions of the sample have already been removed from the sample container after a sufficient incubation period(s) for carrying out one or more assays of interest. The activity station for washing the sample container may include a washing device, such as an irrigator which sprays or otherwise expels fluid into the sample container to rinse the sample container. In certain aspects, the rinsing fluid contains a disinfecting agent, a sterilizing agent, a cleaning agent, or any combination thereof.

Systems of the present disclosure may include an activity site for drying the sample container, e.g., after the sample container has been washed. Such an activity site may include a drying device, such as a device capable of blowing air (e.g., hot air) into the sample container to dry the sample container. In certain embodiments, when the system includes an activity station for washing the sample container and an activity station for drying the sample container, the activity stations may be a single activity station having a washing device and a drying device combined into a singular device.

Any of the activities described herein may be performed within a single activity site, or may be separated and performed at two or more activity sites.

According to certain embodiments, systems of the present disclosure include instructions which, when executed by the processor, cause the system to rotate the sample container through the plurality of activity sites at a predetermined periodic time period. For example, the sample container (e.g., including the sample) may be rotated sequentially from one activity site to a subsequent activity site at a predetermined periodic time period, e.g., every 18-20 seconds, for instance.

In certain aspects, incubating systems of the present disclosure include a plurality of sample containers positioned at the peripheral edge of the rotary platform and spaced approximately equally from each other about a central axis of rotation of the rotary platform. By “spaced equally” or “spaced approximately equally” is meant that the closest distance between any two neighboring sample containers along the peripheral edge of the rotary platform is no less than 80% (e.g., no less than 90%, no less than 95%, or no less than 99%) of the greatest distance between any two neighboring sample containers along the peripheral edge of the rotary platform. As used herein, a sample container is “positioned at a peripheral edge” of the rotary platform when the center of the sample container is positioned within the peripheral (or “outer”) half of the rotary platform with respect to the distance between the center of the platform and the peripheral (or “outer”) edge of the platform nearest the sample container. For example, if the rotary platform is circular and has a radius of 30 cm, a sample container is positioned at a peripheral edge of the platform if the center of the sample container is within 15 cm of the edge of the sample container.

The number of activity sites and/or sample containers may vary. For example, the system may include from 1 to 20 activity sites, e.g., 1, 2, 3, 4, 5, 6, 7, 8, 9, 10, 11, 12, 13, 14, 15, 16, 17, 18, 19 or 20 activity sites. The number of sample containers present on the rotary platform at any one time may be less, the same, or more than the number of activity sites. In certain aspects, the number of sample containers is from 1 to 20 sample containers, e.g., 1, 2, 3, 4, 5, 6, 7, 8, 9, 10, 11, 12, 13, 14, 15, 16, 17, 18, 19 or 20 sample containers present on the rotary platform at any particular time. Furthermore, the length of the predetermined timed interval may vary. For example, in one embodiment, the rotary platform includes 8 sample containers that are rotated 45 degrees through eight activity sites. In another embodiment, the rotary platform includes eight additional sample containers positioned every 45 degrees, but offset from the other 8 sample containers, for a total of 16 sample containers on the rotary platform. In yet another embodiment, the rotary platform includes 4 sample containers rotated every 90 degrees to different activity stations (e.g., 4 different activity stations).

In certain embodiments, multiple sample containers are present at a single activity site. For example, the rotary platform may rotate a doublet, triplet, or quadruplet, etc. of sample containers through each of the activity sites (e.g., a triplet of sample containers rotated 90 degrees between four activity sites). In some instances, a triple dilution system may perform activities for three samples at each activity site. Other sets of sample containers may be implemented in other embodiments—e.g., sets of two, four, five, etc. Furthermore, another number of activity sites may be implemented—e.g., two, three, five, six, etc.

In certain embodiments, an incubating system of the present disclosure includes 8 sample containers positioned about every 45 degrees about a central axis of rotation of the rotary platform. According to these embodiments, the instructions when executed by the processor may cause the system to rotate the rotary platform 45 degrees at a predetermined periodic time period. The predetermined periodic time period may be any suitable periodic time period, including but not limited to, about 15, 16, 17, 18, 19, 20, 21, 22 seconds, or multiples thereof (e.g., 18 seconds or a multiple of 18 seconds).

As described above, the length of the predetermined time interval between rotations may vary. In one embodiment, for example, eight sample containers may be rotated every 18 seconds between activity sites. For example, the sample containers may be rotated at a time interval that is a multiple of 18 seconds, such as 36 seconds, 54 seconds, 72 seconds, 90 seconds, etc. Other timed interval values may also be implemented. The predetermined timed interval may be adjusted to provide various incubation times for measurements. The location of the activity site for the measurement may vary in different embodiments to provide the desired incubation time period—e.g., at the third activity site would provide a 36 second incubation time, at the fourth activity site would provide a 54 second incubation time, etc.

The systems of the present disclosure may be used in a wide variety of applications, e.g., research and/or clinical applications. The systems find use in incubating samples for any assay of interest. For example, the system finds use in incubating samples for assaying (e.g., detecting and/or quantitating) biological materials including DNA, protein, cells (e.g., CD61+, CD3+, CD4+, and/or CD8+cells), and/or clinically relevant analytes such as alkaline phosphatase, bilirubin, carbon dioxide, creatinine, gamma glutamyl transferase (GGT), nitrogen urea, albumin BCG, albumin BCP, ALT, ALT activated, amylase, AST, AST activated, calcium, chloride, cholesterol, CK, direct bilirubin, direct LDL, glucose, HDL, iron, LDH, lithium, magnesium, phosphorus, potassium, sodium, total protein, triglyceride, uric acid, or any combination thereof

In certain aspects, the incubating system is used to incubate a hematology sample for a hematology assay. For example, the system may be a hematology analyzer. A blood sample may diluted and incubated before one or more measurements are performed. The measurements may be performed by optical detectors (e.g., flow cytometry), photometry detectors, impedance detectors, etc. Example measurements include, but are not limited to, identifying, differentiating, and/or counting of particles, cells, or combinations thereof, within the blood sample. For example, in certain embodiments, the sample may include red blood cells (RBC), white blood cells (WBC), platelets (PLC), nucleated red blood cells (NRBC) and/or hemoglobin (Hb), and the incubating system may be configured to incubate the sample and/or perform a qualitative and/or quantitative RBC, WBC, PLC, NRBC and/or Hb assay.

Different measurements may require different incubation times. For example, incubation times (e.g., 30 seconds) may be shorter for RBC and WBC assays than for an incubation time (e.g., 90 second) needed for a retic or reticulocyte (RET) assay. In certain instances, for example, an RBC and WBC measurement may need to be executed within approximately 30-40 seconds because the RBC and WBC in the same diluted sample may change shapes and volume if it is incubated, e.g., for 90 seconds at 40 degrees Celsius. Furthermore, in some instances, the samples may require heating or mixing before a measurement is performed. Other activities may also be performed either before or after the measurements are performed.

### Methods

Also provided by the present disclosure are methods of incubating samples for an assay (e.g., a hematology assay). The methods include rotating a rotary platform to move a sample container that includes a sample through a plurality of activity sites, where the sample container is positioned at a peripheral edge of the platform, and where the plurality of activity sites includes a first activity site and a second activity site. The methods also include extracting a first portion of the sample from the sample container at the first activity site for a measurement based on a first incubation time period, and extracting a second portion of the sample from the sample container at the second activity site for a measurement based on a second incubation time period, the second incubation time period being longer than the first incubation time period.

In certain aspects, the methods of the present disclosure are carried out using a system according to any of the embodiments described in the previous section entitled “Systems”. For example, the steps of the methods may be performed using a system having any of the features described hereinabove, e.g., the types and numbers of activity sites, the devices associated with the activity sites, sample container configurations, predetermined periodic time periods for rotating one or a plurality of sample containers, etc.

According to certain embodiments of the subject methods, the plurality of activity sites includes an activity site for diluting a sample (e.g., a hematology sample), where the method includes diluting a sample at the activity site for diluting a sample. Diluting the sample may include using a diluent suitable for the particular assay of interest. Diluents may include a buffer (e.g., a phosphate or other suitable buffer), a salt, a surfactant, a dye (e.g., a fluorescent dye, such as a cell membrane-permeable and nucleic acid-binding fluorescent dye), an anti-microbial agent, a lysing reagent, any other diluent component useful for the assay of interest, or any combination thereof.

In certain aspects, the plurality of activity sites include an activity site for mixing a diluted sample, where the method includes mixing a diluted sample at the activity site for mixing a diluted sample. The mixing may be carried out, e.g., using a suitable mixing device, such as a rotary stirrer, a vibration type mixer (e.g., a piezo vibrator, etc.), a motor-driven mixer such as a paddle mixer, e.g., either a rotary paddle mixer, an “up-down” motion paddle mixer, or any other suitable mixing device.

According to the methods of the present disclosure, the plurality of activity sites may include one or more activity sites for incubating samples, where the method may include incubating the sample at an ambient temperature of the system, or additionally or alternatively, heating the sample using a suitable heating device, e.g., a hot air bath, a water bath, a heat block, a heating element positioned in sufficient proximity to the sample container to heat the sample, and/or the like.

The plurality of activity sites may include an activity site for washing sample containers, where the subject methods include washing a sample container at the activity site for washing sample containers. The washing may be performed using a washing device, such as an irrigator which sprays or otherwise expels fluid into the sample container to rinse the sample container. The washing may include disinfecting, sterilizing and/or cleaning the sample container, e.g., by including a disinfecting agent, a sterilizing agent, and/or a cleaning agent in the fluid used to wash the sample container.

In certain aspects, the plurality of activity sites includes an activity site for drying sample containers, where the method includes drying a sample container at the activity site for drying sample containers. The drying may be performed using any suitable drying device, such as a device capable of blowing air (e.g., hot air) into the sample container to dry the sample container.

According to certain embodiments, rotating the rotary platform includes rotating the rotary platform to move the sample container through the plurality of activity sites at a predetermined periodic time period.

In certain aspects, the rotary platform includes a plurality of sample containers positioned at the peripheral edge of the rotary platform and spaced equally from each other about a central axis of rotation of the rotary platform. According to this aspect, the rotating may include rotating the rotary platform to move the plurality of sample containers through the plurality of activity sites at a predetermined periodic time period. According to certain embodiments, the rotating includes rotating the rotary platform about 45 degrees at a predetermined periodic time period to move 8 sample containers positioned about every 45 degrees about a central axis of rotation of the rotary platform through eight activity sites at a predetermined periodic time period. The predetermined periodic time period may be any suitable periodic time period, including but not limited to, about 15, 16, 17, 18, 19, 20, 21, 22 seconds, or multiples thereof (e.g., 18 seconds or a multiple of 18 seconds).

### Utility

The systems and methods of the present disclosure find use in a wide variety of applications (e.g., research and/or clinical applications) in which it is useful to incubate one or more samples for an assay of interest. The subject systems and methods finds use in incubating samples for assaying (e.g., detecting and/or quantitating) biological materials including cells (e.g., RBC, WBC, RET, PLT, NRBC, CD61+, CD3+, CD4+, and/or CD8+ cells), DNA, protein, and/or clinically relevant analytes such as alkaline phosphatase, bilirubin, carbon dioxide, creatinine, gamma glutamyl transferase (GGT), nitrogen urea, albumin BCG, albumin BCP, ALT, ALT activated, amylase, AST, AST activated, calcium, chloride, cholesterol, CK, direct bilirubin, direct LDL, glucose, HDL, iron, LDH, lithium, magnesium, phosphorus, potassium, sodium, total protein, triglyceride, uric acid, or any combination thereof. Accordingly, in certain aspects, the incubation systems and methods of the present disclosure find use in (or in combination with) hematology, molecular biology, biochemistry, and/or clinical chemistry assay systems.

The systems and methods of the present disclosure constitute an improvement over previous systems and methods, in that they permit high throughput assaying of samples for which results of two or more assays are desired, and where at least two of the two or more assays require (or are optimally performed using) different incubation times. For example, in the context of a clinical hematology analyzer and prior to the present disclosure, a single-diluted sample from a blood sample in a single dilution method must be incubated for the maximum incubation time. That is, for hypothetical hematology assays A and B, where assay B requires a longer incubation time than assay A, the diluted sample must be incubated for the longer of two incubation times, even though the longer incubation time may be sub-optimal for/detrimental to assay A. As an example in the context of a clinical hematology analyzer, one may wish to perform on a single-dilution sample an RBC/WBC assay having a preferred incubation time of 36 seconds and a reticulocyte assay having a preferred incubation time of 90 seconds. Prior to the present disclosure, both assays would need to be performed after a 90 second incubation, even though this longer incubation is detrimental to the RBC/WBC assay, e.g., on account of the RBCs and WBCs changing shape and/or volume as a result of the longer (90 second) incubation time. The systems and methods of the present disclosure resolve this problem while still enabling high throughput.

The following detailed description of the figures refers to the accompanying drawings that illustrate exemplary embodiments of the systems and methods of the present disclosure. Other embodiments are possible. Modifications may be made to the embodiments described herein without departing from the spirit and scope of the present invention. Therefore, the following detailed description is not meant to be limiting.

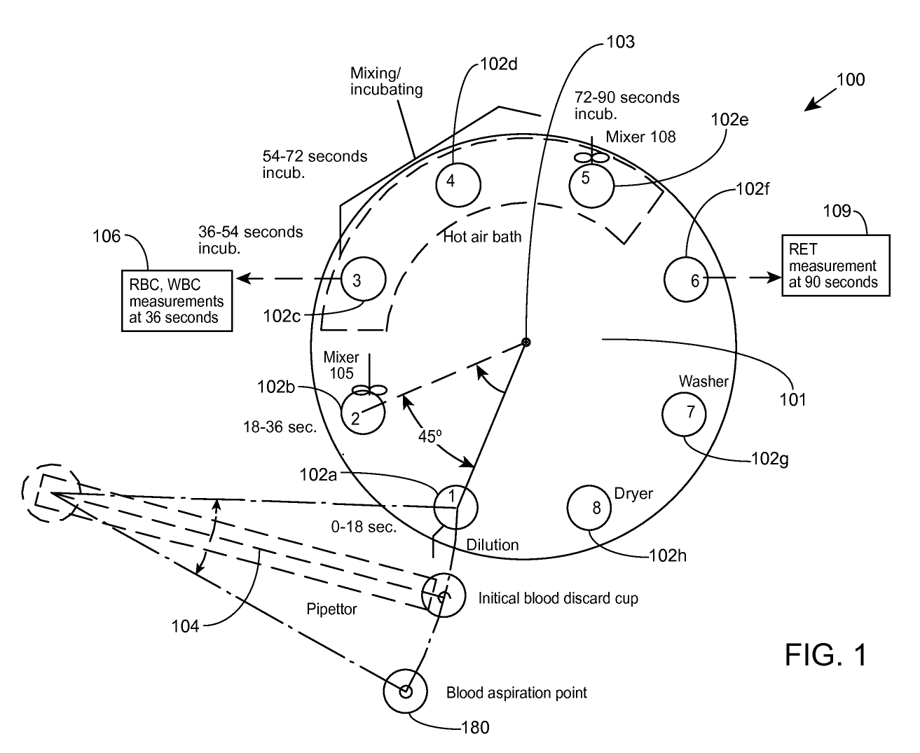

FIG. 1 illustrates a one-dilution incubating system for incubating samples for a hematology assay, according to one embodiment. Incubation system 100 is shown including a rotary platform 101 including eight sample containers 102a, 102b, 102c, 102d, 102e, 102f, 102g, and 102h positioned every 45 degrees about a central axis of rotation 103. The rotary platform 101 is adapted to rotate about the central axis 103. For example, the incubation system may include a motor (not shown) that is operably coupled to the rotary platform 101 to rotate the rotary platform 101 about the central axis 103.

The incubation system 100 also includes activity sites 1, 2, 3, 4, 5, 6, 7, and 8 where various activities are performed. In the instant shown in FIG. 1, each of the sample containers are positioned at respective activity sites 1, 2, 3, 4, 5, 6, 7, and 8. As the rotary platform 101 rotates to a new orientation (e.g., clockwise by 45 degrees), each of the sample containers at activity sites 1, 2, 3, 4, 5, 6, 7, and 8 are rotated to the next clockwise activity site.

Activity site 1 provides a sample within the sample container positioned at activity site 1. For example, incubation system 100 includes a sample delivery device 104 operably positioned at activity site 1 to provide a sample within the sample container. The sample delivery device 104 may be a probe, pipettor, or any other device to deliver sample within the container currently positioned at activity site 1. In one embodiment, as shown, the sample delivery device 104 is rotated to withdraw a blood sample from a sample source 180. In some instances, the delivery device may discard an initial amount of the blood sample before providing a sample within the sample container. In one embodiment, the sample delivery device 104 provides a sample that is diluted—e.g., includes a diluent and/or dye.

Activity site 2 mixes the sample within the sample container that rotated from activity site 1 and currently positioned at activity site 2. For example, incubation system 100 includes a mixing device 105 operably positioned at activity site 2 to mix the sample within the sample container when it arrive at activity site 2.

Activity site 3 extracts a portion of the sample within the sample container rotated from activity site 2 and currently positioned at activity site 3. The sample is extracted for measurements purposes. For example, incubation system 100 may include an extraction device (e.g., suction probe 106) operably positioned at activity site 3 to extract a portion of the sample within the sample container currently positioned at activity site 3.

Activity site 4 incubates the sample within a sample container rotated from activity site 3 and currently positioned at activity site 4. The sample is heated while incubating at activity site 4. For example, incubation system 100 includes a heating device 107 operably positioned at activity site 4 to heat the sample within the sample container. For instance, the heating device 107 may be a hot air bath provided on the underside of the rotary platform 101.

Activity site 5 mixes the sample within a sample container rotated from activity site 4 and currently positioned at activity site 5. For example, incubation system 100 may include another mixing device 108 operably positioned at activity site 5 to mix the sample within the sample container. In the embodiment shown, heating device 107 is also operably positioned at activity site 5 to heat the sample within the sample container.

Activity site 6 extracts a portion of the sample within a sample container rotated from activity site 5 and currently positioned at activity site 6. The portion of the sample is extracted for measurement purposes for instance. For example, incubation system 100 includes another extraction device 109 (e.g., suction probe) operably positioned at activity site 6 to extract a portion of the sample within the sample container. Furthermore, the measurement occurs at activity site 6.

Activity site 7 washes the sample container rotated from activity site 6 and currently positioned at activity site 7. For example, incubation system 100 may include a washing device 110 operably positioned at activity site 7 to rinse the sample container. The washing device 110 may be, for example, an irrigation source to spray liquid, such as water or cleaning solution, into the sample container to rinse out any remaining sample.

Activity site 8 dries the sample container rotated from activity site 7 and currently positioned at activity site 8. For example, incubation system 100 may include a dryer device 111 operably positioned at activity site 8 to dry the sample container. The dryer device 108 may be, for example, an hot air source to blow hot air into the sample container to dry out any remaining liquid from the washing device 110.

### Example Use

Eight mixing cups are on rotary platform 101 at 45 degrees intervals. The rotary platform 101 rotates clockwise by 45 degrees at a predetermined time period of 18 seconds.

At activity site 1, for example, a blood sample is diluted within a sample container. For example, the blood sample may be diluted by a diluent. In some instances, the blood sample may also be diluted with dye solution. For example, the diluted sample may comprise a ratio of 1:35 blood to diluent and dye, such as 37.5 uL blood to 1275 uL diluent and dye, with a total volume of approximately 1313 uL. The example values are provided for exemplary purposes and should not be viewed as limiting.

After 18 seconds, the rotary platform 101 is then rotated 45 degrees clockwise, which moves each sample container to the next clockwise activity site. Thus, for example, the sample container which has the diluted sample from activity site 1, is rotated clockwise to activity site 2. At activity site 2, mixer 105 mixes the diluted sample within the sample container. For example, the mixer 105 may mix the diluted sample for 14 seconds, or any other predetermined time.

At the 36 second mark, the rotary platform 101 is then rotated again 45 degrees clockwise, the sample container at activity site 2 is now at activity site 3. At activity site 3, a portion of the diluted sample from the sample container is extracted by an extraction device, such as a suction probe for instance, for the performance of a measurement at an incubation time of 36 seconds. For example, a suction probe may extract a portion of the diluted sample and transfer it to a flow cell for assaying. In certain embodiments for instance, the assay may comprise a measurement for red blood cell (RBC), platelet (PLT), white blood cell (WBC), also referred to herein as “assaying RBC/PLT/WBC”, or a measurement for RBC and PLT, also referred to as “assaying RBC/PLT”. For example, the suction volume may comprise approximately half of the total volume (e.g., 650 uL of 1313 uL), or other sized portions of the diluted sample as required.

The diluted sample continues to be rotated and is further incubated between activity sites 3 and 5. For example, at the 54 second mark, the sample container is rotated to activity site 4, and at the 72 second mark, the sample container is rotated to activity site 5. In the embodiment shown, the sample is incubated between activity sites 3 and 5 while being subjected to a hot air bath—e.g., positioned under the rotary platform for instance. At activity site 5, the diluted sample is mixed by a mixer 108. After being rotated to activity site 6 at the 90 second mark, a portion (or all) of the remaining diluted sample is then extracted with a suction probe for another measurement at an incubation time of 90 seconds. The sample may be extracted, for example, to a flow cell for measurement analysis.

At the 108 second mark, the sample container is rotated to activity site 7, where any remaining sample in the sample container is discarded and the container rinsed with rinsing solution. For example, a multi-purpose probe sprays liquid in the cup and suction port at the bottom of the probe vacuums the rinsing solution out.

At the 126 second mark, the sample container is rotated to activity site 8, the sample container is dried—e.g., by a polyethylene porous tip and vacuum suction. The sample container is then rotated to activity site 1 to be used for a new dilution.

It should be appreciated that multiple processes may occur simultaneously for different samples. For example, after the first diluted sample is provided at activity site 1 and rotated to activity site 2, the next diluted sample may be provided in the next sample container positioned at activity site 1. At the next rotation, the next sample container to arrive at activity site 1 will receive a diluted sample. This may be repeated for each rotation, and eventually the first sample container will come back around to activity site 1 and a new diluted sample will be provided within it. In this way, a high throughput is achieved. In one embodiment, for example, the 8 sample containers rotating 45 degrees every 18 seconds may achieve a high throughput—e.g., 200 runs per hour.

It should be appreciated that in other embodiments, the predetermined time period between rotations may differ. In one embodiment, the predetermined time period is a multiple of 18 seconds. Furthermore, it should be appreciated that in other embodiments, the position of the activity sites for measurements may be positioned accordingly to provide the appropriate incubation time based on the time between rotations. Furthermore, it should be appreciated that in other embodiments, a different number of sample containers than 8 may be implemented.

In the embodiment discussed above, two assays are performed at activity sites 3 and 6. As multiple samples are being incubated and rotated simultaneously, the two assays at activity sites 3 and 6 are being performed each time for two different samples—e.g., one sample currently at activity site 3 and the other sample currently at activity site 6. In certain embodiments, two optics bench may be used for each assay.

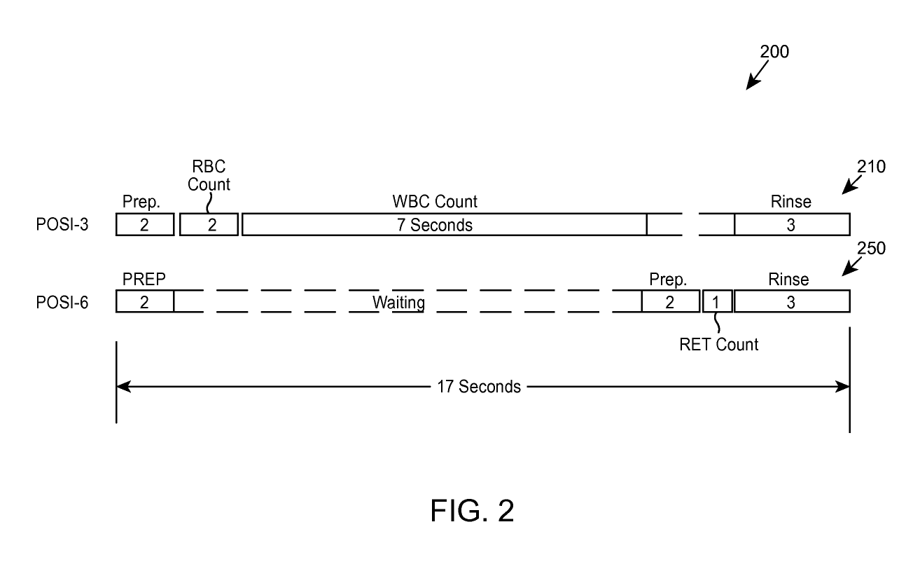

In certain embodiments, a single optics bench may be used for multiple assays. FIG. 2 illustrates a time diagram for an example method in two assays may be performed by a single optical bench. While the predetermined rotation interval for the example is 18 seconds, the rotation of the platform takes some time to get to the next activity site (e.g., 1 second). Thus, the time diagram illustrated represents the 17 seconds in which the rotary platform stays still at each activity site.

In the example shown, time diagram 200 includes a time diagram for a first measurement 210 for the portion of the sample container extracted from the sample container at activity site 3, and a time diagram for a second measurement 250 for the portion of the sample container extracted from the sample container at activity site 6. For example, measurement 210 performs a RBC count and WBC count, while measurement 250 performs a retic (RET) count. In the 18 seconds that the two samples are at activity sites 3 and 6, the optics bench runs the RBC/WBC and the RET samples in a series mode including suction time and rinsing time. As shown, in the first two seconds of the 18 second interval, the samples for measurements 210 and 250 are prepped (e.g., extracted for positioning the flow cell). After the 2 seconds of prep, measurement 210 continues with a RBC count taking 2 seconds, followed a WBC count taking 7 seconds. Measurement 250 waits during this time, but then begins after the 7 second WBC count. For example, after the 7 second WBC count, measurement 250 includes a 2 second prep time (e.g., positions the sample in the flow cell), followed by 1 second to perform a RET count. After, the RET count is performed, the remaining three seconds are used to rinse both staging tubes.

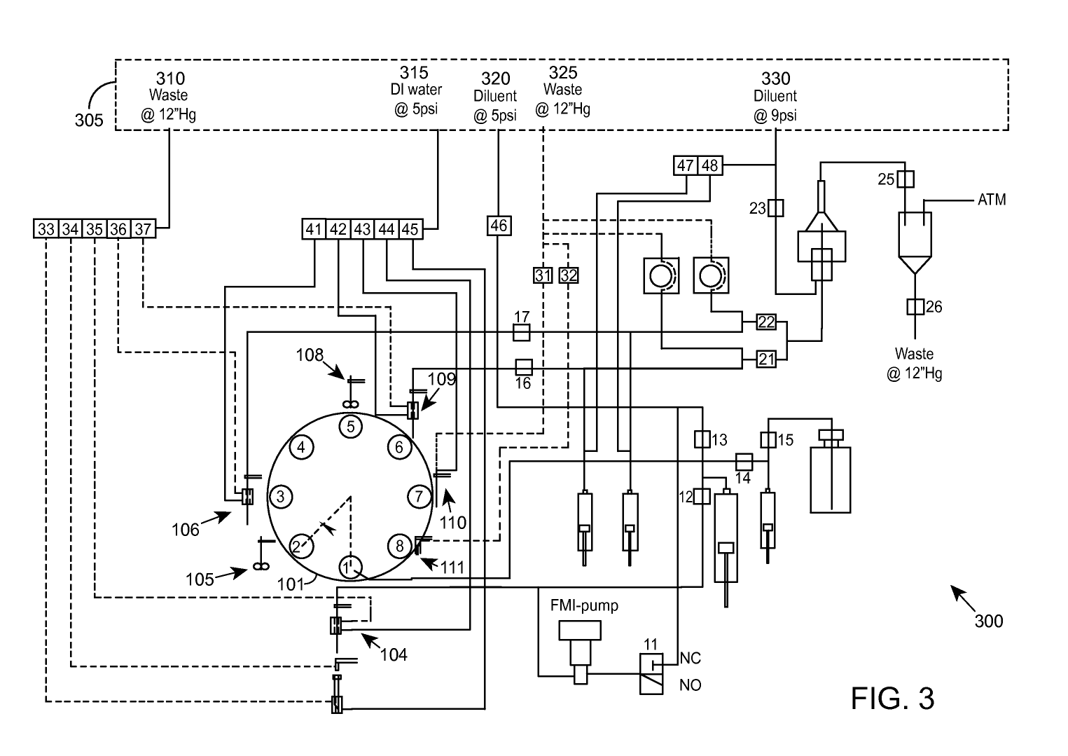

FIG. 3 illustrates a diagram of an example system including the incubation system described in FIG. 1, according to one embodiment. As shown, system 300 is shown including rotary platform 101, sampling device 104, mixers 105 and 108, suction probes 106 and 109, washer 110 and dryer 111, which operate as described above. The system 300 also includes a plurality of valves 11-26, 31-37, and 41-48 operably coupled with conduits between a source panel 305 and the rotary platform 101, sampling device 104, mixers 105 and 108, suction probes 106 and 109, washer 110 and dryer 111, as shown. The source panel 305 includes, for example, ports for waste lines 310 and 325, deionized (DI) water 315, diluent lines 320 and 330. In other embodiments, one or more sources may be separately positioned—i.e., not necessarily part of a single source panel housing all sources and/or source ports.

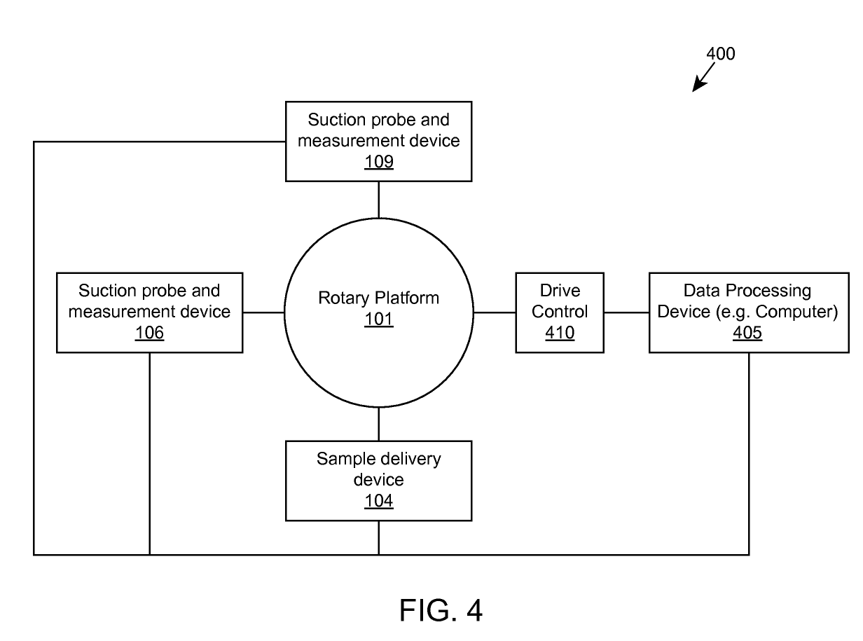

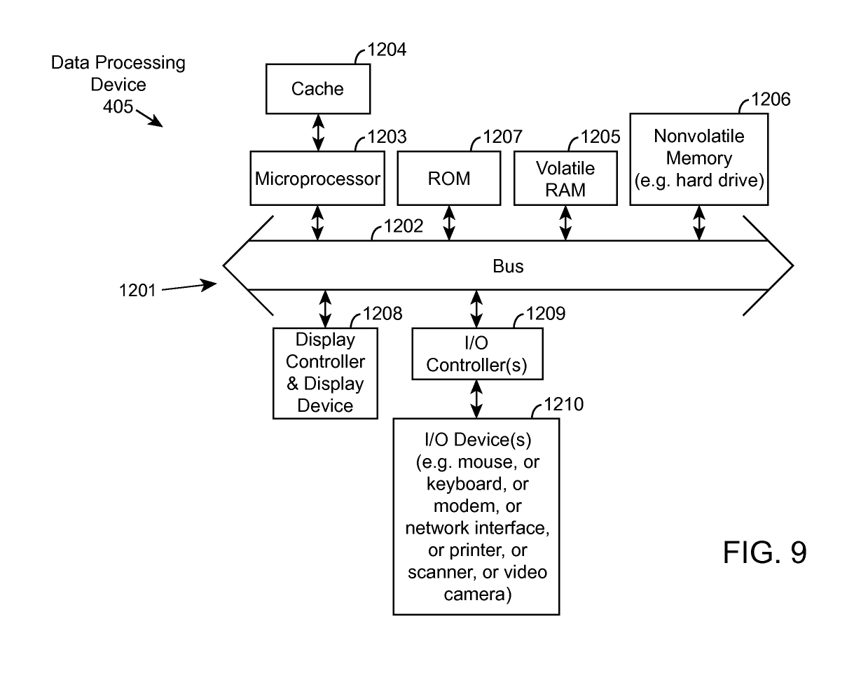

FIG. 4 illustrates a block diagram for an exemplary system including the incubation system described in FIG. 1, according to one embodiment. For example, the system 400 is shown including a data processing device 405 (e.g., a computer) communicably coupled to the rotary platform 410 through a rotational drive control 410. An example embodiment of a data processing device is provided in FIG. 9. The rotational drive control 410 may include, for example, a controller that receives instructions by the computer to activate and deactivate a rotary drive mechanism coupled to the rotary platform through a motor and gears for example. The computer 400 may also be communicably coupled to the sampling device 104, and suction probes 106 and 109 to control the coordination and synchronization of the devices with the rotary platform 101.

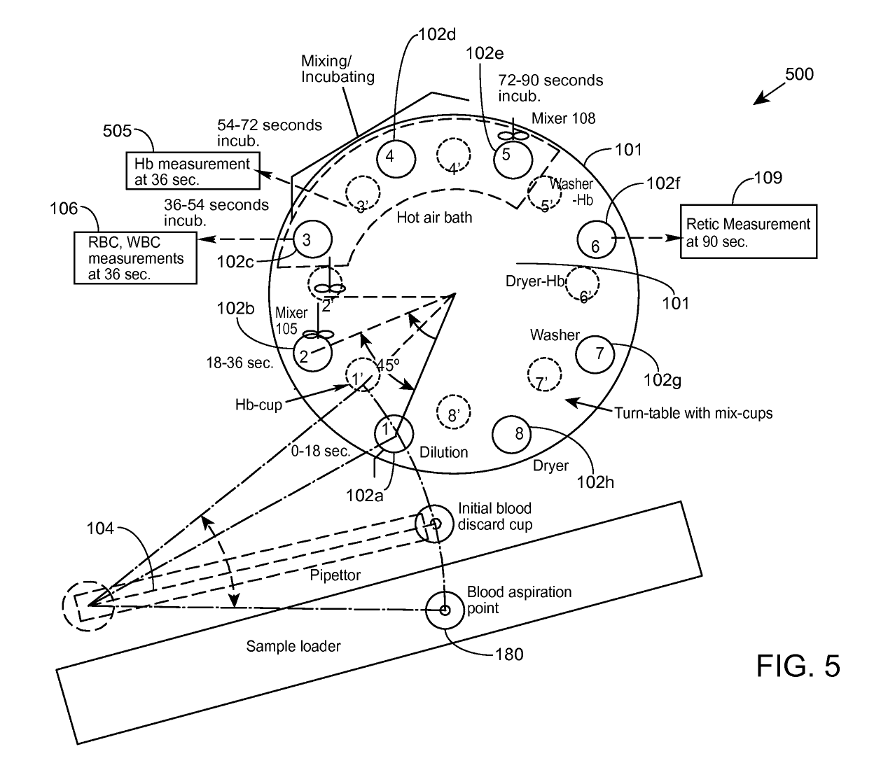

In certain embodiments, additional sampling containers may be implemented. For example, additional sampling containers may be added to the rotary platform 101 described in FIG. 1. FIG. 5 illustrates a system with the rotary platform described in FIG. 1 as well as additional sampling containers for a bulk hemoglobin (HB) measurement besides cell-by-cell HB. For example, the system 500 shown in FIG. 5 includes sample containers at activity sites 1, 2, 3, 4, 5, 6, 7, and 8 as described in FIG. 1, as well as additional sample containers at activity sites 1′, 2′, 3′, 4′, 5′, 6′, 7′, and 8′ for the HB measurement. For the sake of brevity and clarity, the similar features of FIG. 1 and FIG. 5 are not again described in further detail, but previous description is applicable. System 500 also includes a suction probe 505 that extracts some or all of the HB sample to perform a HB measurement simultaneously and in parallel with RBC/WBC measurements from samples taken by suction probe 106.

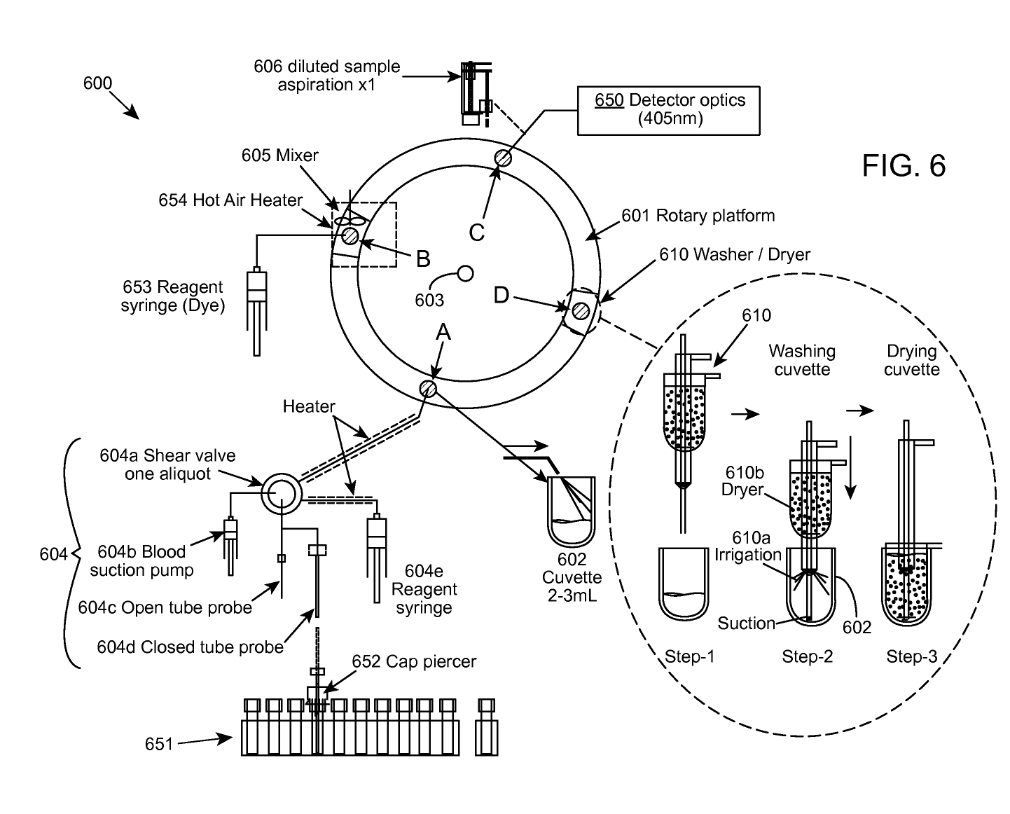

FIG. 6 illustrates a one-dilution incubation system, according to certain embodiments.

Incubation system 600 is shown including a rotary platform 601 including four sample containers positioned at activity sites A, B, C, and D every 90 degrees about a central axis of rotation 603. The example sample container 602 shown is a 2-3 mL cuvette. Other containers may be implemented in other embodiments. The rotary platform 601 is adapted to rotate about the central axis 603. For example, the incubation system may include a motor (not shown) that is operably coupled to the rotary platform 601 to rotate the rotary platform 601 about the central axis 603.

The incubation system 600 also includes activity sites A, B, C, and D where various activities are performed. In the instant shown in FIG. 6, each of the sample containers (e.g., cuvettes) are positioned at respective activity sites A, B, C, and D. As the rotary platform 601 rotates to a new orientation (e.g., clockwise by 90 degrees), each of the sample containers at activity sites A, B, C, and D are rotated to the next clockwise activity station. Again, the time between rotations may vary. In one embodiment, the predetermined time period is 18 seconds.

Activity site A provides a sample within the sample container positioned at activity site A. For example, incubation system 600 includes a sample delivery device 604 operably positioned at activity site A to provide a sample within the sample container. In the embodiment shown, the sample delivery device 604 includes multiple components, such as a shear valve 604a operably coupled to a blood suction pump 604b, probes 604c and 604d (e.g., open tube probe and/or close tube probes), reagent syringe 604e. The pump 604b provides suction for the one or more of the probes 604c and 604d to withdraw sample form the respective source containing the sample (e.g., blood sample). For example, closed tube probe 604d includes a cap piercer 652 to pierce the caps of the set 651 of close tubes containing the blood sample when a blood sample is to be withdrawn. The shear valve 604a dilutes the blood sample with reagent form reagent syringe 604e, and provides one aliquot to activity site A. In some instances, the reagent and diluted sample are heated by a heating element 652 as shown.

After a predetermined time period, the diluted sample at activity site A is rotated to activity site B. At activity site B, the diluted sample is further diluted with dye by reagent syringe 653 and further heated by heating element 654 while mixer 605 mixes the sample.

After another predetermined time period, the sample is rotated to activity site C where some or all of the sample is extracted by extraction device 606 (e.g., aspirator) and a measurement performed on the extracted sample at optical bench 650.

After another predetermined time period, the sample container at activity site C is rotated to activity site D, where the sample container is washed and dried by a washer-dryer 610. An example washer-dryer 610 is also shown in FIG. 6. The washer-dryer 610 includes an irrigator 610A and a dryer 610B. The irrigator 610A is positioned to enter a sample container 602 positioned at activity site D, and rinse the sample container with a rinsing solution. After rinsing, the dryer 610B is inserted within the sample container 602 to provide air (e.g., heated air) to the sample container to dry the sample container.

After another predetermined time period sufficient to wash and dry the sample container at activity site D, the rotary platform is rotated to bring the sample container at activity site D back to activity site A, whereby the process may repeat. Again, it should be appreciated that multiple processes may occur simultaneously for different samples.

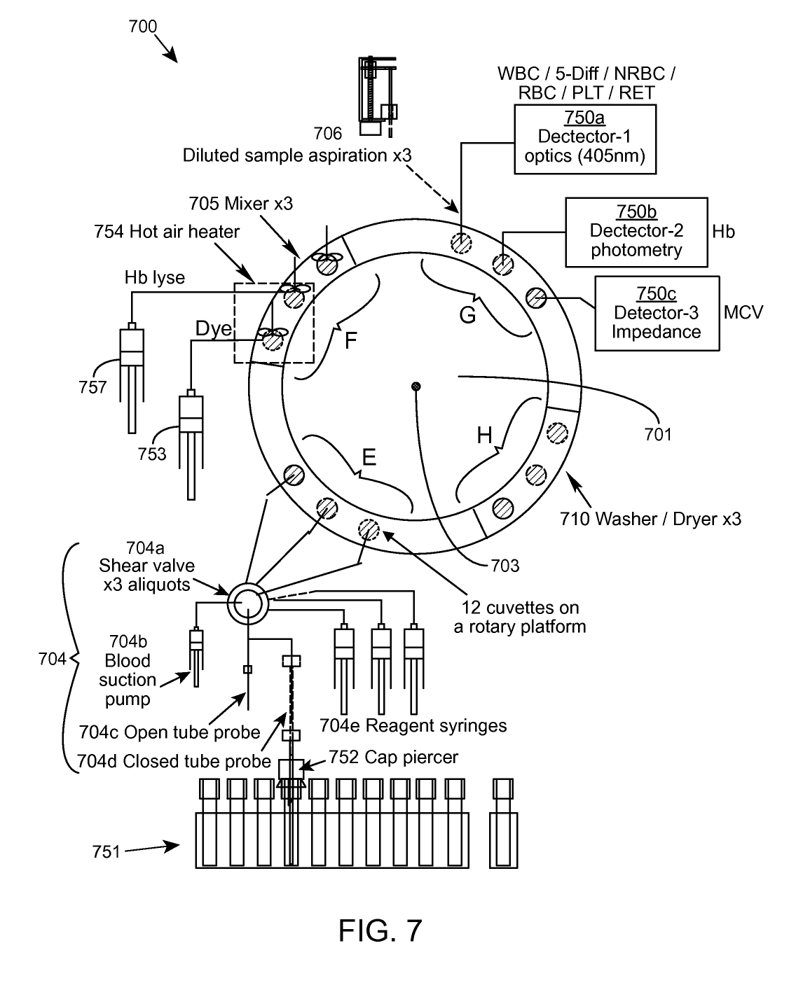

FIG. 7 illustrates a three-dilution incubation system, according to certain embodiments. Incubation system 700 is shown including a rotary platform 701 including twelve sample containers positioned in triplets at activity sites E, F, G, and H every 90 degrees about a central axis of rotation 703. The rotary platform 701 is adapted to rotate about the central axis 703. For example, the incubation system may include a motor (not shown) that is operably coupled to the rotary platform 701 to rotate the rotary platform 701 about the central axis 703.

The incubation system 700 includes activity sites E, F, G, and H where various activities are performed. In the embodiment shown in FIG. 7, a triplet of sample containers are positioned at respective activity sites A, B, C, and D. As the rotary platform 701 rotates to a new orientation (e.g., clockwise by 90 degrees), each of the triplet of sample containers at activity sites E, F, G, and H are rotated to the next clockwise activity station.

Activity site E provides samples within the triplet of sample containers positioned at activity site E. For example, incubation system 700 includes a sample delivery device 704 operably positioned at activity site E to provide the samples within each of the triplet of sample containers. It should be appreciated that the sample delivery device 704 may comprise a single system that sequentially provides a sample within each container in the triplet. Alternatively, the sampling delivery device 704 may include three delivery devices that simultaneously deliver samples within the sample containers.

In one embodiment, the sample delivery device 704 includes multiple components, such as a set of three shear valves 704a with operably coupled to a set of blood suction pump 704b, set of probes 704c and 704d (e.g., open tube probe and/or close tube probes), set of reagent syringes 704e. Pump 704b provides suction for the one or more of the probes 704c and 704d to withdraw samples from the respective sources containing the sample (e.g., blood sample). For example, closed tube probe 704d includes a cap piercer 752 to pierce the caps of the set 751 of close tubes containing the blood sample when a blood sample is to be withdrawn. The shear valves 704a dilute the blood sample with reagent from the set of reagent syringes 704e, and provides three aliquots to three sample containers positioned at activity site E.

After a predetermined time period, the triplet of samples at activity site E are rotated to activity site F. At activity site F, devices 753 and 757 dilute samples with dye and HB lyse, respectively; heating element 754 heats the samples, and three mixers 705 mix the samples.

After another predetermined time period, the triplet of samples are rotated to activity site G where some or all of the samples are extracted by extraction devices 706 (e.g., three aspirator) and measurements performed on the extracted samples at detectors 750A, 750B, and 750C. For example, detector 750 may be an optical detector used to perform WBC/5-Diff/NRBC/RBC/PLT/RET measurements; detector 750B may be a photometry detector used for HB measurement; and detector 750C may measure impedance for a MCV measurement (e.g., using the Coulter method).

After another predetermined time period, the triplet of sample containers at activity site

G are rotated to activity site H, where the sample containers are washed and dried by a washer-dryers 710. After another predetermined time period sufficient to wash and dry the sample containers at activity site H, the rotary platform is rotated to bring the sample containers at activity site H back to activity site E, whereby the process may repeat. Again, it should be appreciated that multiple processes may occur simultaneously for different samples. Furthermore, it should be appreciated that the devices at one or more of the activity stations may be implemented in triplets to simultaneously perform each activity, or alternatively may be performed sequentially with a single device.

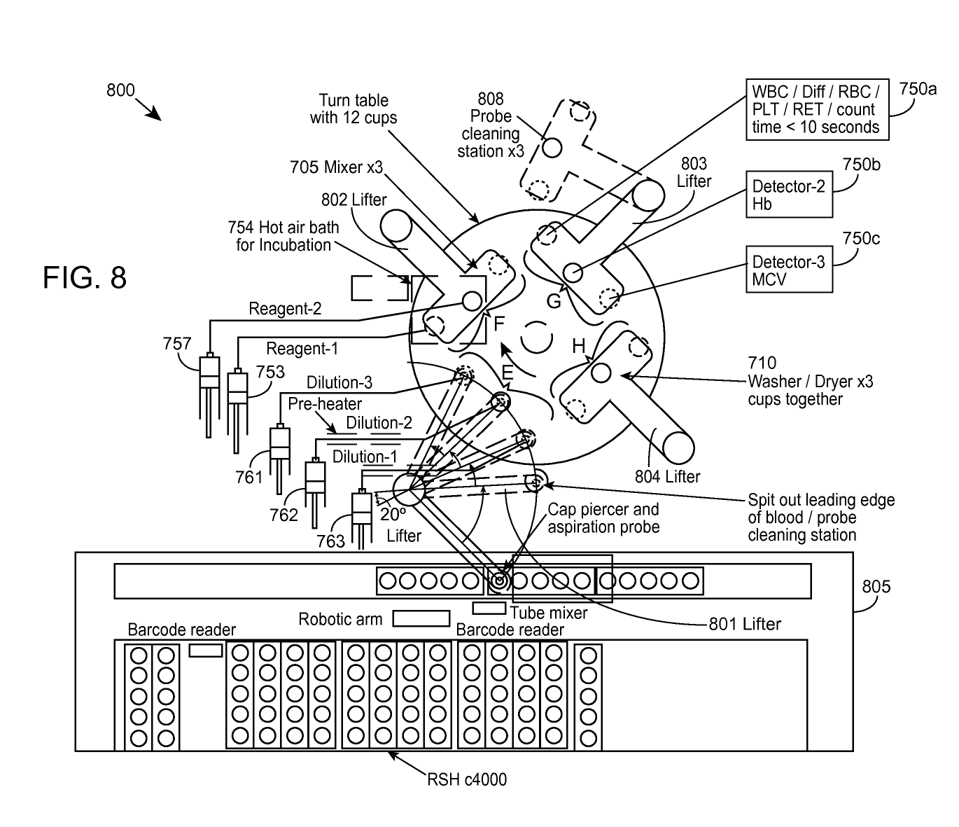

FIG. 8 illustrates the incubation system of FIG. 7, according to one embodiment. The incubation system 800 is shown including lifter arms 801, 802, 803, and 804. Lifter arm 801 is located at activity site E, and may be rotated to collect sample from the sample source 805. A single lifter arm 801 is shown collecting the sample from a sample tube of source 805. The lifter arm 801 may include a suction probe that collects a volume of sample, and when rotated consecutively to the first, second, and third sample containers, dispenses a predetermined amount of blood sample into each sample container of the triplet. Also shown are dilution devices 761, 762, and 763 (e.g., reagent syringes) which are operably coupled to a respective sample container within the triplet of sample containers. Each blood sample from the source may be diluted with the respective dilution devices 761, 762, and 763. Lifter arm 802 is located at activity site F, and includes three mixers which are each inserted into a separate sample of the triplet of sample containers. A hot air bath 754 may be located underneath the rotary platform 701 to provide hot air to the samples. Lifter arm 803 is activity site G, and includes three suction probes that are each used to extract some or all of the sample within a respective sample container for measurements performed at respective detectors 750A, 750B, and 750C. As shown, after the suction probes extract some or all of the samples for measurement, the lifter arm may rotate to a cleaning station 808 to clean the probe to prevent cross contamination. Lifter arm 804 includes a washer-dryer 710 that has three washer-dryers, one for each sample container. For example, each of the washer-dryers may be similar to the washer-dryer 610 shown in FIG. 6.

FIG. 9 illustrates a block diagram of an example data processing device 405 of FIG. 1, according to one embodiment. Embodiments of the present invention may be practiced with various computer system configurations such as hand-held devices, microprocessor systems, microprocessor-based or programmable user electronics, minicomputers, mainframe computers and the like. The embodiments can also be practiced in distributed computing environments where tasks are performed by remote processing devices that are linked through a wire-based or wireless network. FIG. 9 shows one example of data processing system, such as data processing system 405, which may be used with the present described embodiments. Note that while FIG. 9 illustrates various components of a data processing system, it is not intended to represent any particular architecture or manner of interconnecting the components as such details are not germane to the techniques described herein. It will also be appreciated that network computers and other data processing systems which have fewer components or perhaps more components may also be used. The data processing device of FIG. 9 may, for example, be a personal computer (PC), workstation, tablet, smartphone or other hand-held wireless device, or any device having similar functionality. Furthermore, the term “data processing system” may also encompass programmable circuitry programmed or configured by software and/or firmware, or within special-purpose “hardwired” circuitry, or a combination of such forms. Such special-purpose circuitry (if any) can be in the form of, for example, one or more application-specific integrated circuits (ASICS), programmable logic devices (PLDs), field-programmable gate arrays (FPGAs), etc. For example, the data processing device may be in the form of a FPGA including a various modules operably and communicably coupled to one another. For instance, the FPGA may include a module functioning as a processing device, a module functioning as memory, a base line restoration module, a peak detection module, a successive cancellation module, a channel analysis module, etc.

For the example embodiment shown in FIG. 9, the data processing system 1201 includes a system bus 1202 which is coupled to a microprocessor 1203, a Read-Only Memory (ROM) 1207, a volatile Random Access Memory (RAM) 1205, as well as other nonvolatile memory 1206. In the illustrated embodiment, microprocessor 1203 is coupled to cache memory 1204. System bus 1202 can be adapted to interconnect these various components together and also interconnect components 1203, 1207, 1205, and 1206 to a display controller and display device 1208, and to peripheral devices such as input/output (“I/O”) devices 1210. Types of I/O devices can include keyboards, modems, network interfaces, printers, scanners, video cameras, or other devices well known in the art. Some of the I/O devices 1210 may in some instances be coupled to the system bus 1202 through I/O controllers 1209. In one embodiment, the I/O controller 1209 may be communicably coupled to other devices and components of the incubation systems described herein. For example, in one embodiment, a controller may be used to provide power to the motor of drive control 410 to rotate the rotary platform. Similarly, other components such as the suction probes, mixers, washer, dryers, etc., may also include a controller which is communicably coupled to the data processing device 405.

RAM 1205 can be implemented as dynamic RAM (“DRAM”) which requires power continually in order to refresh or maintain the data in the memory. The other nonvolatile memory 1206 can be a magnetic hard drive, magnetic optical drive, optical drive, DVD RAM, or other type of memory system that maintains data after power is removed from the system. While FIG. 9 shows that nonvolatile memory 1206 as a local device coupled with the rest of the components in the data processing system, it will be appreciated by skilled artisans that the described techniques may use a nonvolatile memory remote from the system, such as a network storage device coupled with the data processing system through a network interface such as a modem or Ethernet interface (not shown).

Other embodiments and modifications within the scope of the present disclosure will be apparent to those skilled in the relevant art. Various modifications, processes, as well as numerous structures to which the embodiments of the present disclosure may be applicable will be readily apparent to those of skill in the art to which the present disclosure is directed upon review of the specification. Various aspects and features of the present disclosure may have been explained or described in relation to understandings, beliefs, theories, underlying assumptions, and/or working or prophetic examples, although it will be understood that the present disclosure is not bound to any particular understanding, belief, theory, underlying assumption, and/or working or prophetic example.

It should be understood that some of the techniques introduced above can be implemented by programmable circuitry programmed or configured by software and/or firmware, or they can be implemented entirely by special-purpose “hardwired” circuitry, or in a combination of such forms. Such special-purpose circuitry (if any) can be in the form of, for example, one or more application-specific integrated circuits (ASICS), programmable logic devices (PLDs), field-programmable gate arrays (FPGAs), etc.

Software or firmware implementing the techniques introduced herein may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing took, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc.

Accordingly, the preceding merely illustrates the principles of the systems and methods of the present disclosure. It will be appreciated that those skilled in the art will be able to devise various arrangements which, although not explicitly described or shown herein, embody the principles of the systems and methods of the present disclosure and are included within its spirit and scope. Furthermore, all examples and conditional language recited herein are principally intended to aid the reader in understanding the principles of the systems and methods of the present disclosure and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the systems and methods of the present disclosure, therefore, is not intended to be limited to the exemplary embodiments shown and described herein. Rather, the scope and spirit of systems and methods of the present disclosure is embodied by the appended claims.

That which is claimed is:

1. An incubation system for incubating samples for an assay, the incubating system comprising:
a rotary platform;
a sample container comprising a sample positioned at a peripheral edge of the platform;
a plurality of activity sites comprising at least a first and a second activity site;
a processor; and
a memory comprising instructions when executed by the processor cause the system to:
rotate the rotary platform to move the sample container from the first activity site to the second activity site;
extract a first portion of the sample from the sample container at the first activity site for a measurement based on a first incubation time period; and
extract a second portion of the sample from the sample container at the second activity site for a measurement based on a second incubation time period, the second incubation time period being longer than the first incubation time period.

2. The incubating system of claim 1, wherein the plurality of activity sites comprises an activity site for diluting a sample.

3. The incubating system of claim 2, wherein the system comprises a dilution device for diluting a sample at the activity site for diluting a sample.

4. The incubating system of claim 1, wherein the plurality of activity sites comprises an activity site for mixing a diluted sample.

5. The incubating system of claim 4, comprising a mixing device to mix a sample present in a sample container at the activity site for mixing a diluted sample.

6. The incubating system of claim 5, wherein the mixing device is selected from the group consisting of: a rotary stirrer, a vibration-type mixer, and a motor-driven mixer.

7. The incubating system of claim 1, wherein the plurality of activity sites comprises an activity site for incubating samples.

8. The incubating system of claim 7, wherein the activity site for incubating samples does not comprise a heating device.

9. The incubating system of claim 7, wherein the activity site for incubating samples comprises a heating device.

10. The incubating system of claim 9, wherein the heating device comprises a hot air bath.

11. The incubating system of claim 1, wherein the plurality of activity sites comprises an activity site for washing sample containers and an activity site for drying sample containers.

12. The incubating system of claim 1, wherein the instructions when executed by the processor cause the system to rotate the sample container through the plurality of activity sites at a predetermined periodic time period.

13. The incubating system of claim 1, comprising a plurality of sample containers positioned at the peripheral edge of the rotary platform and spaced equally from each other about a central axis of rotation of the rotary platform.

14. The incubating system of claim 13, comprising 8 sample containers positioned every 45 degrees about a central axis of rotation of the rotary platform, wherein the instructions when executed by the processor cause the system to rotate the rotary platform 45 degrees at a predetermined periodic time period.

15. The incubating system of claim 14, wherein the instructions when executed by the processor cause the system to rotate the rotary platform at a predetermined periodic time period of 18 seconds or a multiple of 18 seconds.

16. The incubating system of claim 1, comprising an extraction device to extract the first portion of the sample from the sample container at the first activity site for a measurement based on a first incubation time period.

17. The incubating system of claim 1, comprising an extraction device to extract the second portion of the sample from the sample container at the second activity site for a measurement based on a second incubation time period.

18. The incubating system of claim 16, wherein the extraction device is a pipetting device.

19. A method of incubating samples for an assay, the method comprising:
rotating a rotary platform to move a sample container comprising a sample through a plurality of activity sites, wherein the sample container is positioned at a peripheral edge of the platform, and wherein the plurality of activity sites comprises a first activity site and a second activity site;
extracting a first portion of the sample from the sample container at the first activity site for a measurement based on a first incubation time period; and
extracting a second portion of the sample from the sample container at the second activity site for a measurement based on a second incubation time period, the second incubation time period being longer than the first incubation time period.

20. The method of claim 19, wherein the plurality of activity sites comprises an activity site for diluting a sample, and wherein the method comprises diluting a sample at the activity site for diluting a sample.

21. The method of claim 19, wherein the plurality of activity sites comprises an activity site for mixing a diluted sample, and wherein the method comprises mixing a diluted sample at the activity site for mixing a diluted sample.

22. The method of claim 21, wherein the mixing is performed by a mixing device selected from the group consisting of: a rotary stirrer, a vibration-type mixer, and a motor-driven mixer.

23. The method of claim 19, wherein the plurality of activity sites comprises an activity site for incubating samples.

24. The method of claim 23, wherein the activity site for incubating samples does not comprise a heating device.

25. The method of claim 23, wherein the activity site for incubating samples comprises a heating device.

26. The method of claim 25, wherein the heating unit comprises a hot air bath.

27. The method of claim 19, wherein the plurality of activity sites comprises an activity site for washing sample containers, and wherein the method comprises washing a sample container at the activity site for washing sample containers.

28. The method of claim 19, wherein the plurality of activity sites comprises an activity site for drying sample containers, and wherein the method comprises drying a sample container at the activity site for drying sample containers.

29. The method of claim 19, wherein the rotating comprises rotating the rotary platform to move the sample container through the plurality of activity sites at a predetermined periodic time period.

30. The method of claim 19, wherein the rotary platform comprises a plurality of sample containers positioned at the peripheral edge of the rotary platform and spaced equally from each other about a central axis of rotation of the rotary platform, and wherein the rotating comprises rotating the rotary platform to move the plurality of sample containers through the plurality of activity sites at a predetermined periodic time period.

31. The method of claim 30, comprising rotating the rotary platform 45 degrees at a predetermined periodic time period to move 8 sample containers positioned every 45 degrees about a central axis of rotation of the rotary platform through 8 activity sites at a predetermined periodic time period.

32. The method of claim 31, wherein the predetermined periodic time period is 18 seconds or a multiple of 18 seconds.
